(19) United States
(12) Patent Application Publication
(10) Pub. No.: US 2024/0407739 A1
(43) Pub. Date: Dec. 12, 2024

(54) MEDICAL DEVICES HAVING MODIFIABLE FUNCTIONALITY AND METHODS OF MANAGING MEDICAL DEVICE FUNCTIONALITY VIA APPLICATIONS

(71) Applicants: Shruti Abhijit GADGIL, Bengaluru, KA (IN); Hui HUI, Wuxi, Jiangsu (CN); Dan LI, Shanghai (CN); Menglu JIANG, Shanghai (CN); GE Precision Healthcare LLC, Waukesha, WI (US)

(72) Inventors: Shruti Gadgil, Bangalore (IN); Hui Hui, Wuxi (CN); Dan Li, Shanghai (CN); Menglu Jiang, Shanghai (CN)

(21) Appl. No.: 18/700,271

(22) PCT Filed: Oct. 11, 2021
(86) PCT No.: PCT/CN2021/123082

§ 371 Date: Apr. 11, 2024

(51) Int. Cl. A61B 5/00; G06F 9/48; G06F 9/54

(52) U.S. Cl. CPC A61B 5/7435; A61B 5/746; G06F 9/48; G06F 9/542

(57) ABSTRACT

A medical device for a patient. The medical device includes a memory system configured to store available applications. A computing system is configured to execute the available applications stored in the memory system, where each of the available applications corresponds to a function performed via the medical device. An applications management module is executable by the computing system to add an additional application to the available applications stored in the memory system, where subsequent execution of the additional application by the computing system causes the medical device to perform the function corresponding thereto. A display device is configured to display the available applications stored in the memory system that are available for execution.

### FIELD

The present disclosure generally relates to medical devices and methods of managing medical devices to modify functionality thereof.

### BACKGROUND

Patient monitors are essential medical devices, which are often used within a hospital environment. These patient monitors constitute a high-cost investment for such medical facilities. Additionally, the technology within the medical field evolves very quickly. At the same time, the design and development cycle for patient monitors is very long, and thus potential customers may be lost (and customer needs left unmet) in the interim between advances in technology and/or medicine, and the release and purchase of a new product incorporating these advances.

The presently disclosed medical devices and methods enable users to modify the functionality of medical devices beyond what was available at the initial purchase, specifically via management over the applications installed thereon. Moreover, the presently disclosed solutions provide for this modification to be easy and on an as-needed basis, using existing platforms (e.g. GE Healthcare's® B1x5 M/P Platform). The result is a faster deployment of applications providing new or improved functionality, and the flexibility of using only those applications needed, rather than awaiting the release of a new product.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One example of the present disclosure generally relates to a medical device for a patient. The medical device includes a memory system configured to store available applications. A computing system is configured to execute the available applications stored in the memory system, where each of the available applications corresponds to a function performed via the medical device. An applications management module is executable by the computing system to add an additional application to the available applications stored in the memory system, where subsequent execution of the additional application by the computing system causes the medical device to perform the function corresponding thereto. A display device is configured to display the available applications stored in the memory system that are available for execution.

In certain examples, the applications management module is further executable to remove a removable application from the available applications stored in the memory system. In further examples, the removable application was previously added as the additional application, and the applications management module automatically removes the removable application a predefined period after the removable application was added to the available applications in the memory system.

In certain examples, the applications management module is configured such that the additional application is non-executable after a predefined period. In further examples, the applications management module is configured such that the predefined period is adjustable.

In certain examples, one of the available applications is configured to generate a notification for a user, and wherein the applications management module is configured to display a notification indication on the display device indicating when the notification is generated by the one of the available applications. In further examples, the notification generated by the one of the available applications is one of a plurality of notifications, and wherein the notification indication includes a number of the plurality of notifications generated. In further examples, the notification indication is selectable to display the notification generated by the one of the available applications, and wherein selecting the notification indication to display the notification causes the applications management module to discontinue displaying the notification indication. In further examples, the notification is an alarm.

In certain examples, the medical device is a monitoring device configured to measure physiological data from the patient.

In certain examples, the applications management module is configured to download the additional application to the memory system of the medical device via a pool of applications stored in a remote database separate from the medical device. In further examples, the applications management module is configured such that the available applications stored in the memory system are executable without access to the remote database. In further examples, the applications management module is further configured to measure a usage of the additional application. In further examples, the applications management module is configured to communicate the usage measured for the additional application to the remote database.

Another example according to the present disclosure generally relate to a method of managing functions available for a medical device. The method includes storing available applications on a memory system and configuring a computing system to execute the available applications stored in the memory system, where each of the available applications corresponds to a function performed via the medical device. The method further includes providing an applications management module that is executable by the computing system to add an additional application to the available applications stored in the memory system, where subsequent execution of the additional application by the computing system causes the medical device to perform the function corresponding thereto. The method further includes configuring a display device to display the available applications stored in the memory system that are available for execution.

Certain examples further include configuring the applications management module to automatically remove a removable application from the available applications stored in the memory system a predefined period after the removable application was added to the available applications in the memory system.

Certain examples further include configuring the applications management module such that the additional application is non-executable a predefined period after being added to the memory system. Further examples provide that one of the available applications is configured to generate a notification for a user, where the applications management module is configured to display a notification indication on the display device indicating when the notification is generated by the one of the available applications. Further examples include configuring the applications management module to download the additional application to the memory system of the medical device via a pool of applications stored in a remote database separate from the medical device, where the available applications stored in the memory system are executable without access to the remote database.

Another example according to the present disclosure generally relates to a patient monitoring device that includes a memory system configured to store available applications. A computing system is configured to execute the available applications stored in the memory system, where each of the available applications corresponds to a function performed via the medical device. An applications management module is executable by the computing system to download an additional application from a remote database to the available applications stored in the memory system, where subsequent execution of the additional application by the computing system causes the medical device to perform the function corresponding thereto. A display device is configured to display the available applications stored in the memory system that are available for execution. The applications management module is configured to display a notification indication on the display device indicating when the notification is generated by the one of the available applications. The applications management module is configured such that the available applications stored in the memory system are executable without access to the remote database. The applications management module is configured such that the additional application is non-executable a predefined period after being downloaded.

Various other features, objects and advantages of the disclosure will be made apparent from the following description taken together with the drawings.

### BRIEF DESCRIPTION OF THE DRAWINGS

The present disclosure is described with reference to the following drawings.

FIG. 1 is perspective view of a medical device according to the present disclosure in-use for monitoring a patient;

FIG. 2 is a schematic view of an example of a control system such as may be incorporated within the medical device, remote database, and/or external devices;

FIG. 3 is a schematic view of an example of a data structure and communication scheme for the medical device and remote database of FIG. 1, and additional external devices;

FIG. 4 is a screenshot of a medical device according to the present disclosure showing a standard monitoring display;

FIG. 5 is an example of a flow chart for managing the functionality of a medical device according to the present disclosure;

FIG. 6 is a screenshot of the medical device of FIG. 4 showing a quick access panel for accessing an applications management module according to the present disclosure;

FIG. 7 is a screenshot of the medical device of FIG. 4 showing another method for accessing the applications management module according to the present disclosure;

FIG. 8 is a screenshot of the medical device of FIG. 4 showing the selection of an example of an application within the applications management module; and

FIGS. 9-10 are screenshots showing a mini-bar along with a number of notifications associated with the application selected in FIG. 8 as notifications occur.

### DETAILED DISCLOSURE

The present inventors have recognized that the product development cycle for patient monitoring devices (and other medical devices) is much slower than that of the applications that run on them. As such, there is an unnecessary gap in timing between when a new function is developed, and when that function becomes available through the release of the next medical device on which the corresponding application is loaded. In certain examples, this timing gap is on the order of years, causing a great disconnect between customer (and patient) needs and solutions from manufacturers responsive to those needs.

Additionally, the need for particular applications on medical devices varies greatly based upon the intended use of that medical device, even within a given model number. For example, the same patient monitoring device may be used in a first case to primarily monitor blood oxygenation in a patient with a breathing condition, and in a second case in an emergency room to diagnose whether a patient is experiencing a heart attack. In this case, a specialized application used for diagnosing heart-characteristics (e.g., analysis of ST waves) is not needed for a medical device situated in the pulmonary care unit, for example. Other non-limiting examples include monitoring basic vital signs and parameters in low acuity (e.g., in a general ward), advanced metabolic monitoring in critically ill patients, advanced cardiac applications for diagnosis and monitoring myocardial infarctions in cardiac units, and/or gas monitoring for ventilated patients.

Furthermore, the inventors have recognized that needs for applications vary over time, and that the addition of further applications may be desirable on an existing medical device. Similarly, the inventors have recognized an unmet need for offering different pricing models for obtaining and/or using applications, whereby systems presently known in the art simply embed this within the cost of the medical device. Once again, the inclusion of specialized, adult cardiac-care applications unduly increases the cost of a medical device used for neonates, or monitoring a patient undergoing a periodontic procedure, for example.

Accordingly, the present inventors have developed the presently disclosed medical devices and methods for managing the functionality of medical devices to address these unmet needs and provide further functionality.

Patient monitors are essential medical devices, including within a hospital environment. However, these patient monitors also constitute a high-cost investment for such medical facilities. Technology within the medical field is evolving very fast, and therefore new monitors are being made available to the market quite frequently. At the same time, the design and development cycle for patient monitors is very long, and thus potential customers may be lost (and customer needs left unmet) in the interim between advances in technology and/or medicine and release of a new product incorporating these advances.

The presently disclosed solutions enable users to modify the functionality of medical devices via management over the applications installed thereon. Moreover, the presently disclosed solutions provide for this modification to be easy and on an as-needed basis, using existing platforms (e.g. GE Healthcare's® B1x5 M/P Platform). In other words, the present disclosure provides for programmable medical devices that enable the installation and execution of new software, features, and functionality, versus static medical devices on which these changes cannot be made. The result is a faster deployment of applications providing new or improved functionality, and the flexibility of using only those applications needed, rather than awaiting the release of a new product.

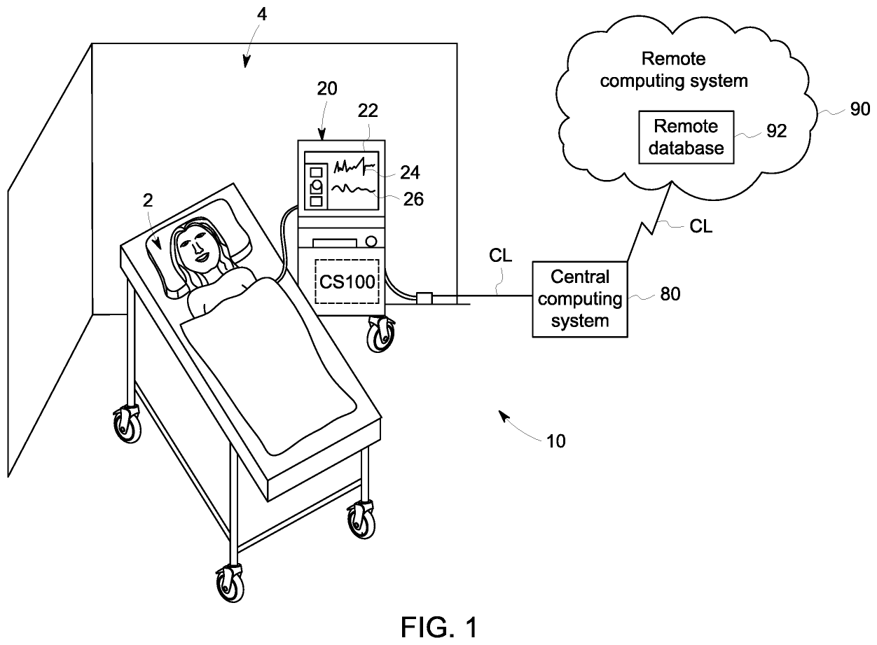

FIG. 1 shows a patient 2 positioned on a bed within a room 4 of a medical facility, such as a hospital or medical clinic. The patient 2 is operatively connected to a medical device 20, such as GE Healthcare's B1x5 M/P Platform, such that the caregiver may monitor various physiological parameters in a manner known in the art. The medical device 20 includes a computing system CS100, which controls operation of the medical device 20. Additional information regarding the computing system CS100 is provided below. The medical device 20 shown here further includes a display device 22 that serves as a graphical user interface (for example, a touch-screen GUI 24). Waveforms 26 are produced on the display device 22 for the different physiological parameters being monitored. Additional information may also be provided on the display device 22, which also enables a user to configure the medical device 20 in a customary manner, and also in the manner discussed further below in accordance with the present disclosure.

The medical device 20 is part of a greater system 10, which includes a central computing system 80 operatively connected to the medical device 20 via a communication link CL in a manner presently known in the art. In the configuration shown, the central computing system 80 is further connected to a remote computing system 90, which may be accessible as a cloud computing device over the internet, for example. The remote computing system 90 of the present system 10 further includes, either directly or indirectly, a remote database 92, as discussed further below.

It should be recognized that the central computing system 80 and the remote computing system 90 may be incorporated into a single device, whether positioned locally (e.g., within a hospital) or remotely. Likewise, it should be recognized that the elements of the central computing system 80, the remote computing system 90, and the medical device 20 may be further combined or subdivided from the examples discussed herein while preserving the same function.

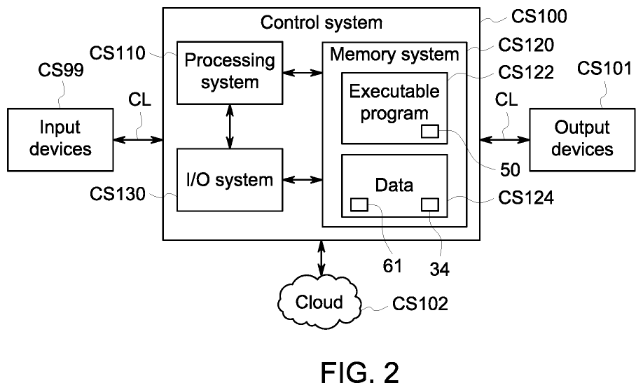

FIG. 2 depicts an example of a control system CS100 such as may be incorporated within the medical device 20 of FIG. 1. The same or similar structure may also or alternatively be provided as part of the central computing system 80, and/or remote computing system 90. Certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely examples, which may be direct or indirect, and may follow alternate pathways.

In certain examples, the control system CS100 communicates with each of the one or more components of the system 10 via a communication link CL, which can be any wired or wireless link. The control system CS100 is capable of receiving information and/or controlling one or more operational characteristics of the system 10 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the system 10. Moreover, the communication link CL lines are meant only to demonstrate that the various control elements are capable of communicating with one another, and do not represent actual wiring connections between the various elements, nor do they represent the only paths of communication between the elements. Additionally, the system 10 may incorporate various types of communication devices and systems, and thus the illustrated communication links CL may in fact represent various different types of wireless and/or wired data communication systems.

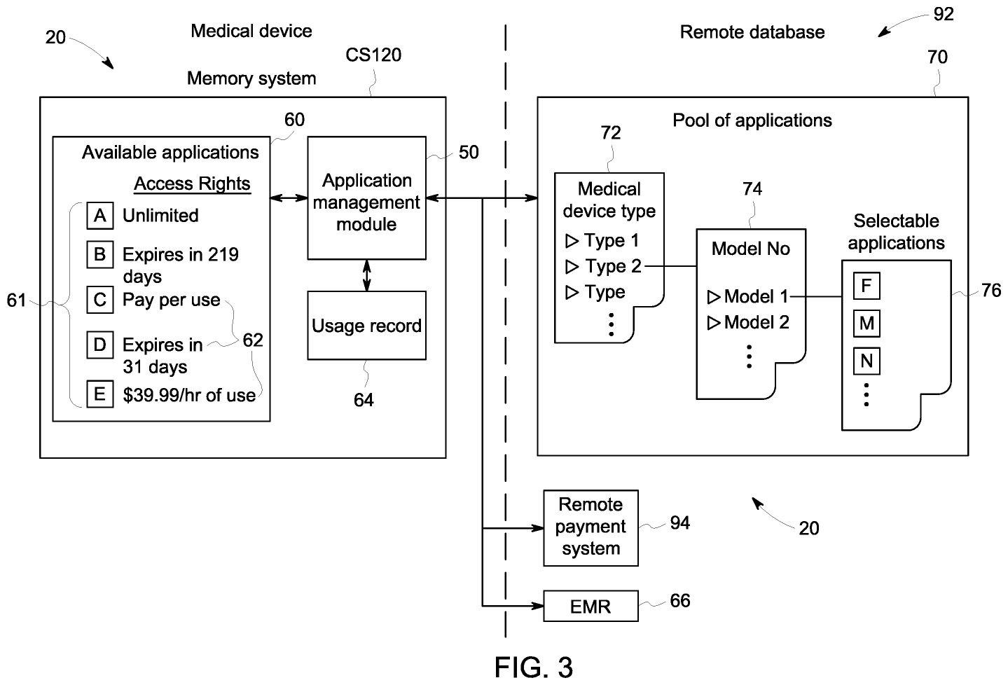

The control system CS100 may be a computing system that includes a processing system CS110, memory system CS120, and input/output (I/O) system CS130 for communicating with other devices, such as input devices CS99 (e.g., sensors and other devices connected to the medical device 20) and output devices CS101 (e.g., the central computing system 80, remote computing system 90, an Electronic Medical Record (EMR 66, see FIG. 3), remote payment system 94 (see FIG. 3), and/or other external devices such as smart phones or tablets), either of which may also or alternatively be stored in a cloud 102. The processing system CS110 loads and executes an executable program CS122 from the memory system CS120, accesses data CS124 stored within the memory system CS120, and directs the system 10 to operate as described in further detail below.

The processing system CS110 may be implemented as a single microprocessor or other circuitry, or be distributed across multiple processing devices or sub-systems that cooperate to execute the executable program CS122 from the memory system CS120. Non-limiting examples of the processing system include general purpose central processing units, application specific processors, and logic devices.

The memory system CS120 may comprise any storage media readable by the processing system CS110 and capable of storing the executable program CS122 and/or data CS124. The memory system CS120 may be implemented as a single storage device, or be distributed across multiple storage devices or sub-systems that cooperate to store computer readable instructions, data structures, program modules, or other data. The memory system CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

FIG. 3 shows an example of a data structure for the medical device 20 and remote database 92, along with communication flows therebetween (and with a remote payment system 94 and EMR 66). The memory system CS120 of the medical device 20 stores an application management module 50, which may be comprised of hardware and/or software executable by the processing system CS110. As discussed further below, the application management module 50 is configured to manage which individual applications are available for use or execution on the medical device 20, and the process of adding and/or removing (and paying for) these applications. In certain examples, applications are automatically removed based on the status or characteristics of a user account. For example, icons to execute applications (or the executable files themselves) may be removed from the GUI or from the memory of the medical device 20 when the application management module 50 determines that a user has closed their account, or that the user account is inactive (e.g., having depleted a pre-determined number or time usage, or a term of access). In another example, applications associated with one type of medical device 20 or use environment may be added and/or removed when the user account becomes re-associated with another type of medical device 20 or environment (e.g., the user account is changed from an emergency room environment to a neonatal care environment, or from being within a network of a large hospital with unlimited access to a single-device configuration with its own access rights and characteristics).

In other examples, the list of available applications stored in the memory system, applications that are executable, and/or applications available for download vary by a profile of a user using the medical device, for example an intensivist versus a nurse versus a bedside clinician. In addition, or in the alternative, the executability of applications may vary based on a level of access the user has within the EMR or other electronic records systems (e.g., for therapeutic applications).

In other examples, the executability of applications may vary based on the diagnosis of the patient. For example, the patient's diagnosis (e.g., a SNOMED code or ICD-10 code) may be inputted via barcode scanner when the patient is admitted to the medical device, such as a bedside monitor. This diagnosis information may also or alternatively be used to make suggestions of available applications based on the specific diagnosis. In certain examples, the medical device includes (and/or communicates with an external device that includes) machine learning and/or artificial intelligence algorithms that learn which applications are executed most, and/or which applications are most impactful in directing the diagnosis, treatment, and recovery of the patient, which is then used to inform the executability and/or recommendations of available applications for use.

In certain examples, usage information of the available applications is also tracked by the medical device and can be used to decide which applications to download or retain within the memory system. For example, the medical device may advise the user or an administrator that a particular application has not been used in a certain number of weeks, for a certain number of patients for which that application is intended, or a percentage of patients for which the application is used (which may again be specific to only patient's having relevant diagnoses). This allows the user or administrator to make informed decisions of which applications to remove, for example to save space within the memory system. In licensing arrangements, this may also enable the administrator to save money by avoiding fees for unnecessary or underutilized applications.

The memory system CS120 further stores a collection of available applications 60, whereby execution of individual applications 61 therein causes the different functions to be performed for the medical device 20. In one example to follow, an intraoperative hypotension (IOH) application is configured to analyze incoming data for the medical device 20 to detect and generate an alarm when hypotension is occurring (e.g., when the Mean Arterial Pressure (MAP) of the patient falls by at least 20% during a procedure). In this example, the IOH application may be started when the procedure starts, providing a baseline for the starting blood pressure of the patient for later comparison. In other examples, the IOH application is configured to accept an inputted starting blood pressure, and/or to designate a starting point, (e.g., a pulse pressure variation baseline value at the beginning of a fluid resuscitation assessment).

It should be recognized that the additional functions performable by the medical device via execution of additional applications cover a broad range. For example, one application may provide for a lung recruitment procedure that causes the valves of a ventilator to perform differently. In other example, the application may regulate an infusion pump rate, such as to adjust flow rates based on the patient and/or measurements detected by the medical device. Another application may adjust the frequency for performing non-invasive blood pressure (NIBP) measurements based on changing needs of the patient, the administration of medications, or other factors. Another application may provide recommendations to the user for gathering additional information or conducting further examination, such as suggesting the number of ECG leads to use for collecting cardiac information (e.g., 5-lead versus 12-lead ECG monitoring).

With continued reference to FIG. 3, additional applications may be added to the available applications 60 on the medical device 20 via the application management module 50. The applications management module 50 is connected to a remote database 92 where these additional applications may be accessed, and/or downloaded to the medical device 20 from, for example via selection from the GUI24 of the display device 22. The remote database 92 may be configured similarly to an applications store for Apple® or Android® smartphones, for example, to add functionality to the medical device 20 in a similar manner. For example, the application management module 50 allows a user to browse a pool of applications 70 stored in the remote database 92 over the internet, to be downloaded locally to the medical device 20 for subsequent offline use. This is particularly advantageous for medical devices 20 used in developing nations, where the devices may be used outside of a facility with internet connectivity, and/or where power or connectivity are frequently interrupted.

In the example of FIG. 3, the pool of applications 70 is structured so as to show the operator only a subset of all possible applications stored therein, and specifically only selectable applications configured to work for the medical device type 72 and model number 74 of the medical device 20 in question. In this manner, the operator of a patient monitoring device need not see or be required to scroll through individual application offerings only for imaging devices or infant warmers, for example. Additional examples of inputs for limiting the listing of applications for selection include a type of ICU where the medical device is deployed (e.g., Adults versus Neonatal versus Pediatric), based on patient diagnosis (e.g., SNOMED CT or ICD-10 codes), based on a procedure being performed on the patient (e.g., CPT code driven), based on patient sex, and/or based on clinical care pathway guidance.

Selecting one or more of the selectable applications 76 in the remote database 92 then causes those applications to join the list of available applications 60 stored locally in the memory system CS120 of the medical device 20. Additionally, the system 10 is configured such that downloading (and/or accessing) one of the selectable applications 76, the transaction is communicated to a remote payment system 94 such that a corresponding invoice may be generated for billing the clinician or facility. For example, accepting the terms to download Application F from the selectable applications 76 may cause the remote payment system 94 to trigger an invoice for $5,000. In other examples, such as for Application E stored in the available applications 60 of the medical device 20, actual use of Application E is monitored and recorded within a usage record 64 also stored in the memory system CS120, which triggers the corresponding communication to the remote payment system 94 in real-time or on a periodic basis.

In this manner, an operator may add new applications to the available applications 60 of a medical device 20 without obtaining a new medical device 20. It should be recognized that the available applications 60 may also or alternatively be stored remotely from the medical device 20, for example within a similar memory system within the central computing system 80. In this case, an administrator may purchase or acquire access rights to new applications for multiple medical devices 20 at a time, which each communicate with the central computing system 80 for execution as needed. For example, an administrator may license one of the selectable applications 76 for all ventilators in a given facility, wing, or floor at that same time (e.g., whereby the usage record may also or alternatively maintain records at this level).

With continued reference to FIG. 3, the database of available applications 60 further includes access rights 62 information for each of the individual applications 61. In the example shown, Application A has rights for unlimited use (e.g., was included with the medical device 20 purchase, and/or was previously purchased outright), Applications B and D were provided as trials or purchased as a limited lease that expire in 219 days and 31 days, respectively, Application C is executable as a pay-per-use application, and Application E is executable for $39.99 per hour of use.

As discussed above, the duration and/or instances of use for using each of the individual applications 61 is maintained in the usage record 64 and communicated to the remote payment system 94 for invoicing consistent with the corresponding access rights 62. The present inventors have recognized that configuring the individual applications 61 to have differing payment options provides flexibility to caregivers. For example, an individual application 61 that would rarely be used by a given facility may be financially unjustified to purchase outright, but may be a valuable option as a pay-per-use or pay-per-time service offering. This also allows medical devices 20 kept as backups to be fully functional if eventually deployed into use, but without having to pay for application licenses for devices that sit idle.

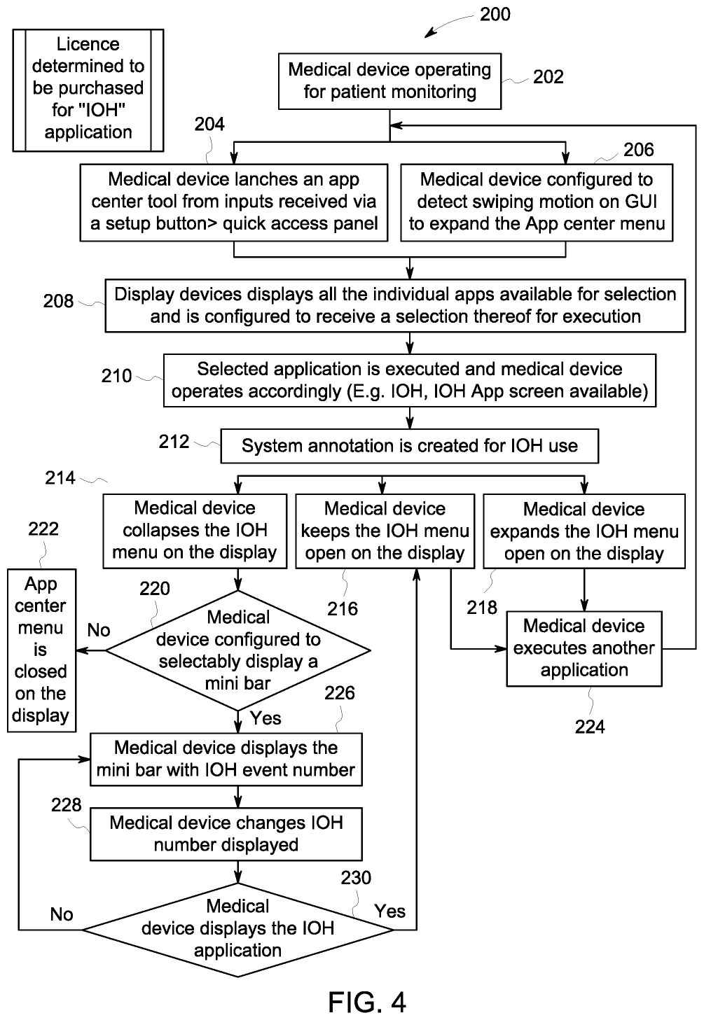

FIG. 4 is a process flow for an example of a method 200 of managing functions available for a medical device 20, such as that previously shown in FIG. 1. In this example, the applications management module 50 will have determined that a license has been purchased or otherwise obtained for an “IOH” application. The process begins with the medical device operating for patient monitoring in step 202. The medical device 20 is configured to launch an App Center tool (also referred to as the application management module 50 either by detecting the selection of a setup button in a quick access panel of the GUI 24 (step 204), or by detecting motions (e.g., swiping right) on the display device 22 (step 206). In step 208, the display device displays all of the individual applications that are enabled or selectable for use on the medical device (from among the available applications 60 thereon), and detects a selection thereof for execution. The medical device then operates or otherwise performs the functions associated with the executed applications in step 210, which in the present example also causes the system 10 to generate an annotation indicating that the given application has been started (step 212). This annotation is particularly helpful in cases in which changes in physiological parameters are detected overtime, whereby the start of the application's use establishes the baseline for subsequent comparison.

Once the application has been started, the medical device is configured to operate in a number of different manners. Step 214 provides for the medical device the menu of the application once executed, allowing the application to continue running in the background. Step 216 provides that the medical device can keep the application menu open (and thus the process returns to step 202), and step 218 that the medical device is also configured so as to expand the application's menu on the display device for use and advanced options therein (step 224). It should be recognized that the advanced options, and functionality in general, or each of the available applications vary in the manner known in the art, and are thus are not explicitly described herein.

With continued reference to FIG. 4, if the menu is collapsed in step 214, the medical device 220 is configured to selectably display a mini-bar, which may further display notifications relating to the executed application running in the background (step 226). The medical device is also configured to be able to close the application management module menu altogether in step 222. Additional discussion of the mini-bar is provided below. If the medical device determines to display the mini-bar in step 220, changes in the notifications related to the executed application may be displayed as a number of notifications that have occurred within that application (step 228). For example, the notifications may be shown as a number of how many alarm conditions have been triggered within that application. The medical device is further configured to allow selection of the application within the mini-bar in step 230 to open the menu corresponding thereto, thereby allowing the user to investigate the basis for the notifications, change alarm limits, or otherwise use the application (and thus returning to step 216).

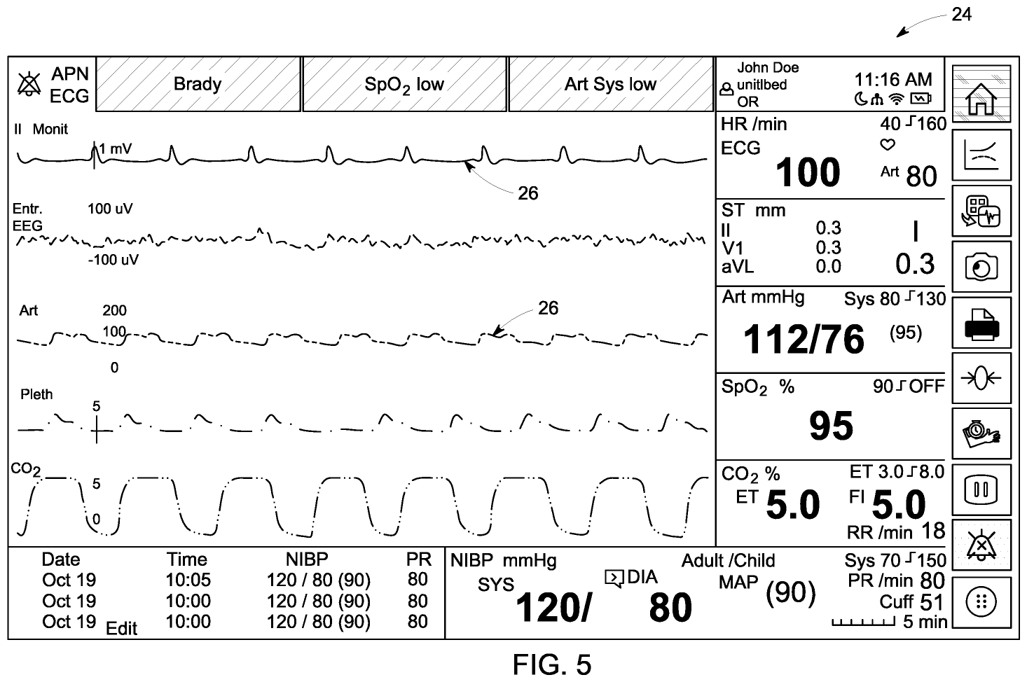

FIG. 5 shows an example of a GUI 24 for a patient monitoring device as the medical device 20, which in this case is displaying various physiological data, including waveforms 26. This information and the waveforms 26 may be native to the medical device 20, and/or be provided as the output of an application presently being executed thereon (including those specifically obtained or accessed through the application management module discussed above).

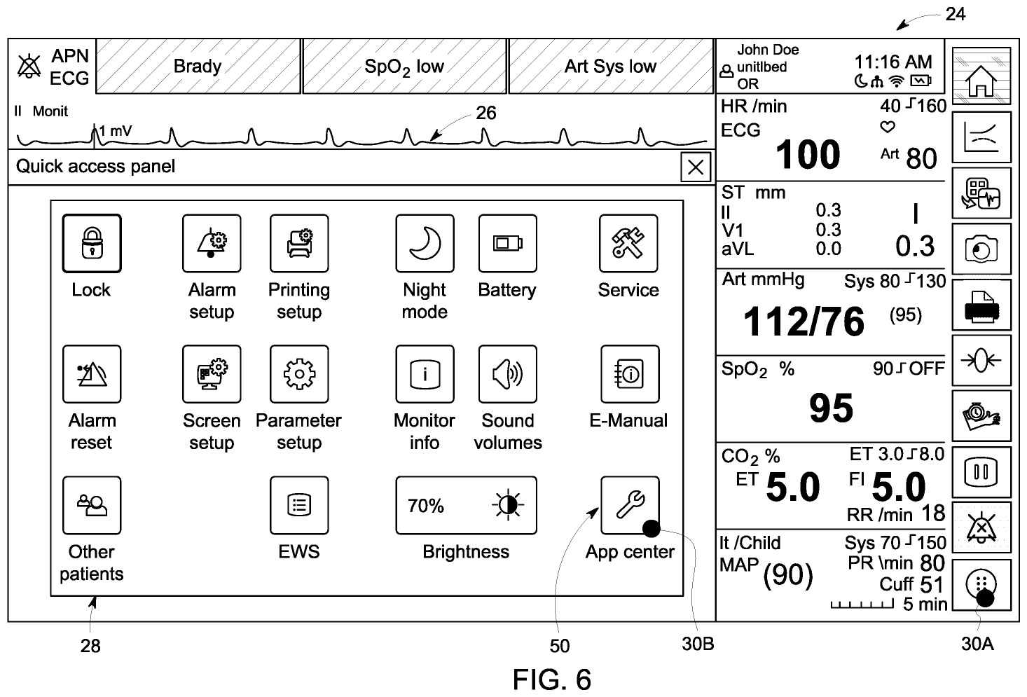

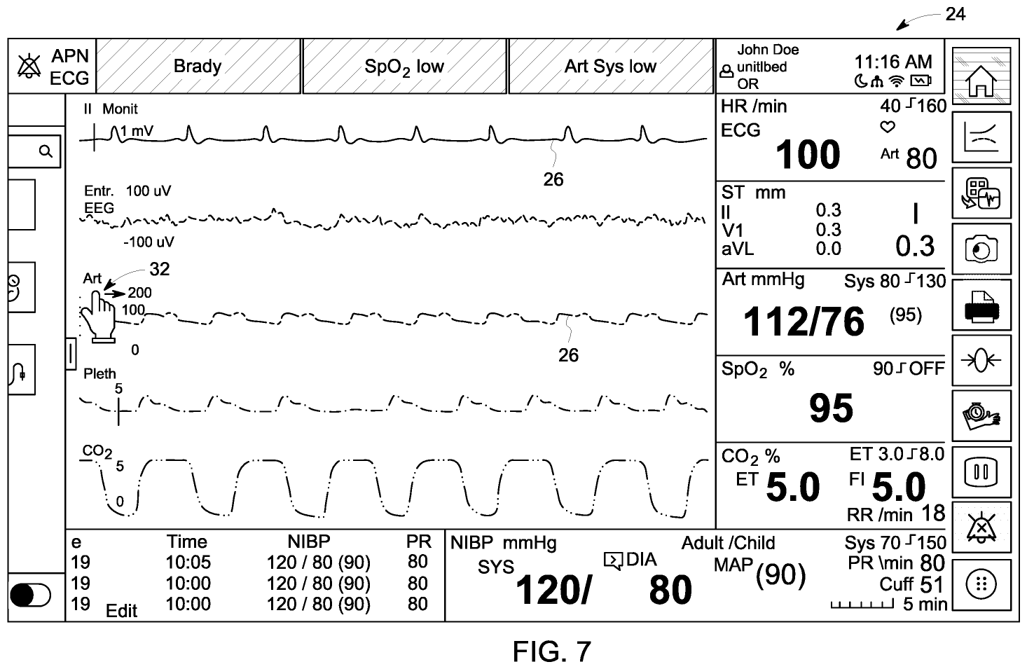

FIGS. 6 and 7 show two methods for opening the application management module 50 discussed above. Specifically, FIG. 6 shows a first method in which a first selection 30A of a button to launch a quick access panel 28 within the GUI 24, followed by a second selection 30B for a button representing the application management module 50, which subsequently opens and is discussed further below. It should be recognized that the quick access panel 28 may provide for making different settings selections above and beyond launching the application management module 50, such as accessing volume settings, brightness settings, and/or the like.

The medical device 20 is also configured such that swiping right (first selection 30A in FIG. 7) within the waveform 26 area causes the application management module 50 to open, or in certain examples, to open the menu of an individual application 61 corresponding to that waveform 26. For example, swiping right on a waveform 26 depicting arterial blood pressure may automatically launch the menu for an application either producing that waveform 26, or providing various functions based on the data reflected within that waveform 26. In certain examples in which a given waveform 26 may have multiple corresponding individual applications 61, swiping to the right may give a list of options for which application to open the menu corresponding thereto, and/or may simply default to opening the application management module 50.

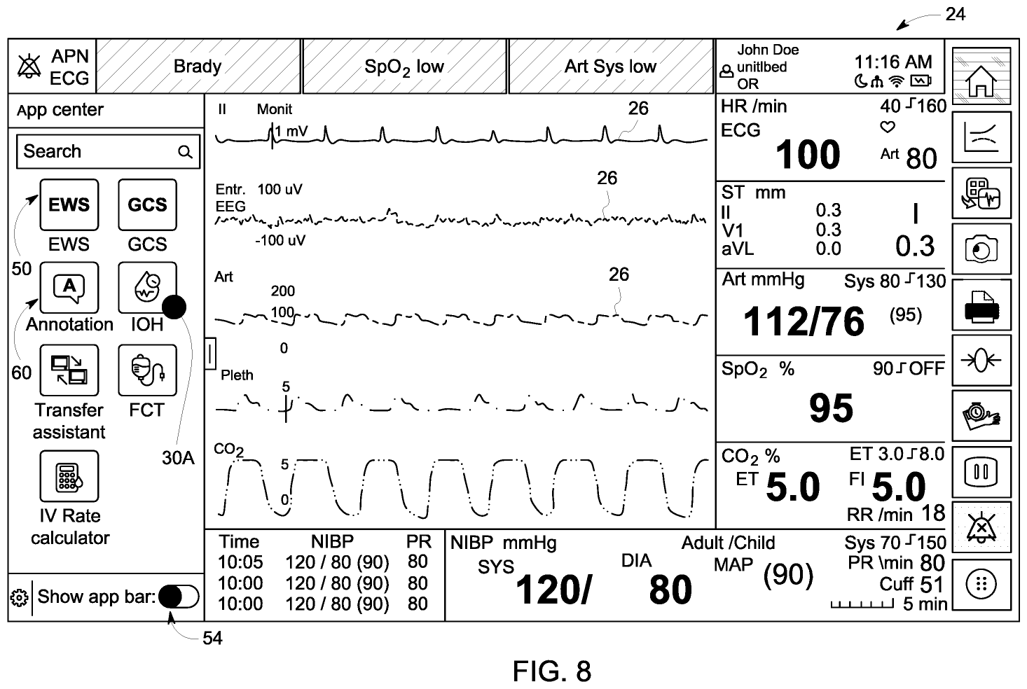

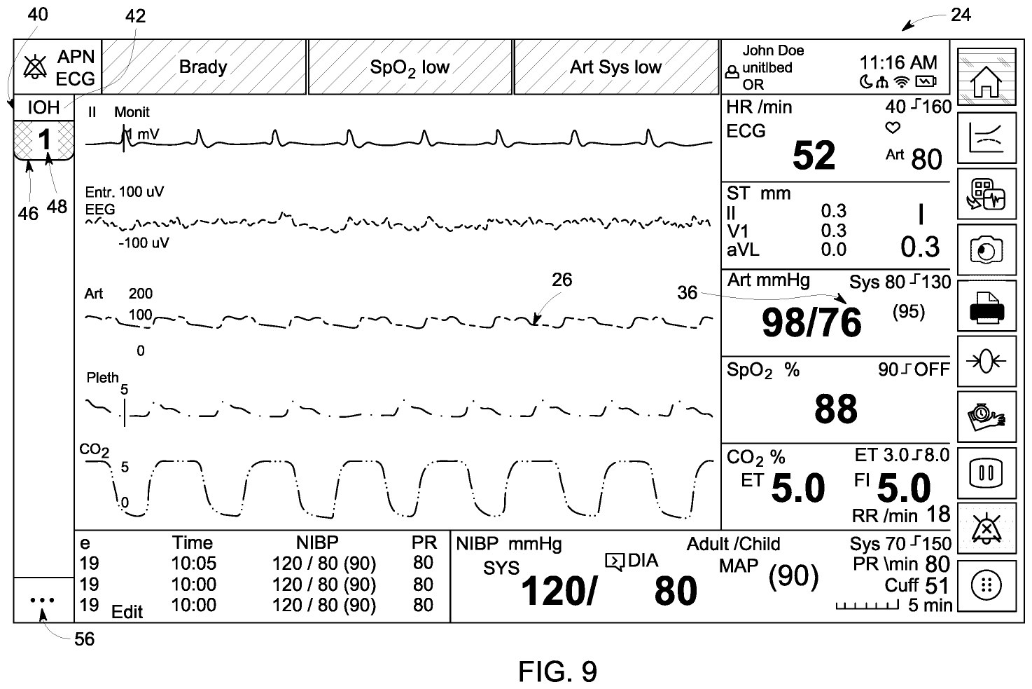

FIG. 8 shows the applications management module 50 menu of available applications 60 open. In this case, an icon labeled as “IOH” corresponds to the intraoperative hypotension application discussed above, which can be executed (or its menu accessed if already running) by selection thereof, for example via the first selection 30A shown. By selecting the radio button 54 within the application management module 50, the GUI 24 transitions to displaying the mini-bar 40 of FIG. 9. Selection of the ellipses 56 in FIG. 9 returns to the application management module 50 of FIG. 8

The mini-bar 40 of FIG. 9 is configured to provide a quick reference to the user for any notifications provided by the various available applications 60 despite them being effectively minimized, and within a minimal footprint of the GUI 24. The medical device 20 may be configured such the mini-bar 40 can be opened by swiping right on the waveform 26, as shown in FIG. 7. With continued reference to FIG. 9, in certain examples, a heading 42 is shown for every individual application 61 presently running on the medical device 20. In other configurations, only the individual applications 61 in which a notification has been generated have a heading 42 in the mini-bar 40.

In the example of FIG. 9, the heading 42 is labeled to indicate that is corresponds to the IOH application, which is running on the medical device 20. In conjunction with this heading 42 is a notification 44, which may be a colored bar or other indicator provided below the heading 42. In the example of FIG. 9, a notification may be generated by the application from the diastolic blood pressure falling to a certain value 36 beyond the allowed limit of a baseline or triggering limit (which may be user-defined within a menu, or a fixed value for example). For example, an “Arterial Systolic Low” alarm may be triggered when the value is determined to be <100 mm of Hg. Similarly, “SpO2 Low” alarm may be triggered when the SpO2 value is <90, and a “Brady” alarm triggered when Heart Rate (HR) value is 60 beats per minute. The notification 44 may be configured such that the coloration corresponds to the severity of the underlying notification (red indicating a serious health condition detected, orange indicating a condition of moderate urgency, and yellow indicating a poor incoming signal quality, for example).

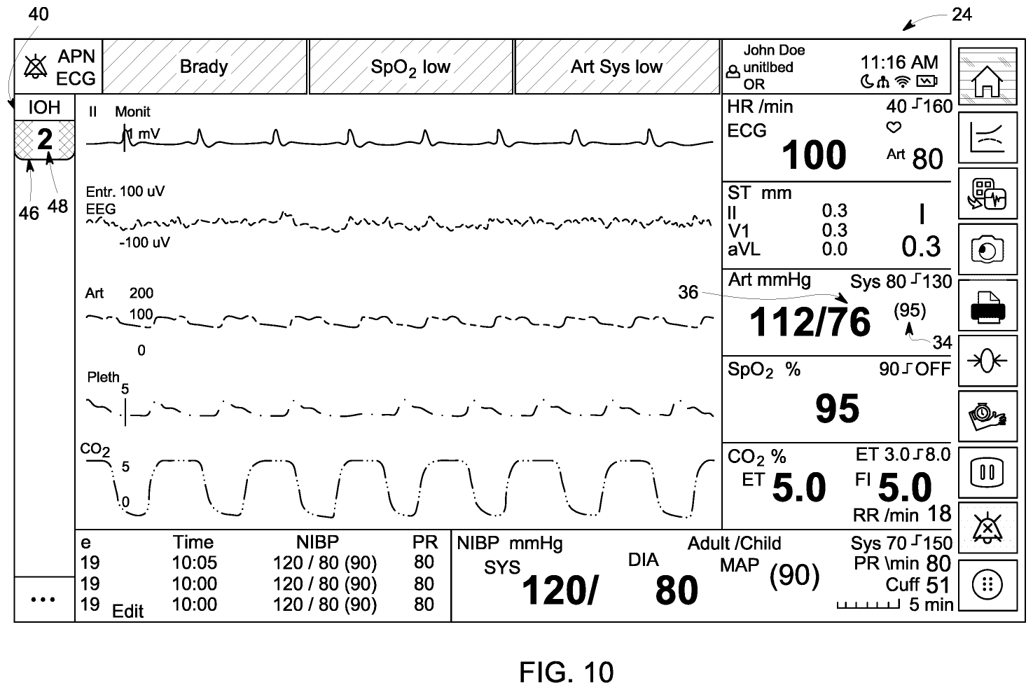

In the example shown in FIG. 9, a numeric indicator 48 is also provided with the notification 44, which here shows a number of notifications that have been triggered by the underlying application since the user last opened the menu corresponding to that application. Transitioning to FIG. 10, the numeric indicator 48 has increase to “2”, indicating that the alarming condition has remained for two notification cycles, a second alarm trigger has occurred (e.g., low and high alarms), and/or other notifications presently known in the art for a given application. This allows the operator to quickly ascertain how much activity has occurred and requires review, particularly for individual applications 61 that have been running in the background.

The functional block diagrams, operational sequences, and flow diagrams provided in the Figures are representative of examples of architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A medical device for a patient, the medical device comprising:
a memory system configured to store available applications;
a computing system configured to execute the available applications stored in the memory system, wherein each of the available applications corresponds to a function performed via the medical device;
an applications management module executable by the computing system to add an additional application to the available applications stored in the memory system, wherein subsequent execution of the additional application by the computing system causes the medical device to perform the function corresponding thereto; and
a display device configured to display the available applications stored in the memory system that are available for execution.

2. The medical device according to claim 1, wherein the applications management module is further executable to remove a removable application from the available applications stored in the memory system.

3. The medical device according to claim 2, wherein the removable application was previously added as the additional application, and wherein the applications management module automatically removes the removable application a predefined period after the removable application was added to the available applications in the memory system.

4. The medical device according to claim 2, wherein the applications management module is configured such that the additional application is non-executable after a predefined period.

5. The medical device according to claim 4, wherein the applications management module is configured such that the predefined period is adjustable.

6. The medical device according to claim 1, wherein one of the available applications is configured to generate a notification for a user, and wherein the applications management module is configured to display a notification indication on the display device indicating when the notification is generated by the one of the available applications.

7. The medical device according to claim 6, wherein the notification generated by the one of the available applications is one of a plurality of notifications, and wherein the notification indication includes a number of the plurality of notifications generated.

8. The medical device according to claim 6, wherein the notification indication is selectable to display the notification generated by the one of the available applications, and wherein selecting the notification indication to display the notification causes the applications management module to discontinue displaying the notification indication.

9. The medical device according to claim 6, wherein the notification is an alarm.

10. The medical device according to claim 1, wherein the medical device is a monitoring device configured to measure physiological data from the patient.

11. The medical device according to claim 1, wherein the applications management module is configured to download the additional application to the memory system of the medical device via a pool of applications stored in a remote database separate from the medical device.

12. The medical device according to claim 11, wherein the applications management module is configured such that the available applications stored in the memory system are executable without access to the remote database.

13. The medical device according to claim 1, wherein the applications management module is further configured to measure a usage of the additional application.

14. The medical device according to claim 13, wherein the applications management module is configured to communicate the usage measured for the additional application to the remote database.

15. A method of managing functions available for a medical device, the method comprising:
storing available applications on a memory system;
configuring a computing system to execute the available applications stored in the memory system, wherein each of the available applications corresponds to a function performed via the medical device;
providing an applications management module that is executable by the computing system to add an additional application to the available applications stored in the memory system, wherein subsequent execution of the additional application by the computing system causes the medical device to perform the function corresponding thereto; and
configuring a display device to display the available applications stored in the memory system that are available for execution.

16. The method according to claim 15, further comprising configuring the applications management module to automatically remove a removable application from the available applications stored in the memory system a predefined period after the removable application was added to the available applications in the memory system.

17. The method according to claim 15, further comprising configuring the applications management module such that the additional application is non-executable a predefined period after being added to the memory system.

18. The method according to claim 15, wherein one of the available applications is configured to generate a notification for a user, and wherein the applications management module is configured to display a notification indication on the display device indicating when the notification is generated by the one of the available applications.

19. The method according to claim 15, further comprising configuring the applications management module to download the additional application to the memory system of the medical device via a pool of applications stored in a remote database separate from the medical device, wherein the available applications stored in the memory system are executable without access to the remote database.

20. A patient monitoring device comprising:
a memory system configured to store available applications;
a computing system configured to execute the available applications stored in the memory system, wherein each of the available applications corresponds to a function performed via the medical device;
an applications management module executable by the computing system to download an additional application from a remote database to the available applications stored in the memory system, wherein subsequent execution of the additional application by the computing system causes the medical device to perform the function corresponding thereto; and
a display device configured to display the available applications stored in the memory system that are available for execution;
wherein the applications management module is configured to display a notification indication on the display device indicating when the notification is generated by the one of the available applications;
wherein the applications management module is configured such that the available applications stored in the memory system are executable without access to the remote database; and
wherein the applications management module is configured such that the additional application is non-executable a predefined period after being downloaded.
